(19) United States
(12) Patent Application Publication
(10) Pub. No.: US 2007/0282208 A1
(43) Pub. Date: Dec. 6, 2007

(54) Mobile computing device with integrated medical devices

(76) Inventors: Bob Jacobs, Portland, OR (US); Isaac Simpson, Beaverton, OR (US)

Correspondence Address: DARBY & DARBY P.C., P.O. BOX 770, Church Street Station, New York, NY, 10008-0770, US

(21) Appl. No.: 11/447,298

(22) Filed: Jun. 6, 2006

(51) Int. Cl. A61B 5/02

(52) U.S. Cl. 600/485

(57) ABSTRACT

The invention relates to a device for use by medical professionals. The device comprises a chassis comprising a motherboard comprising a central processing unit; the motherboard further comprising at least one graphics and memory controller and at least one input/output controller, wherein the input/output controller communicates with at least one wireless device controller; and at least one wireless peripheral device configured to acquire medical data relating a vital sign of a patient and communicate the medical data via the wireless device controller. The device is characterized as a tablet-style personal computer having a sealed chassis that is resistant to liquid penetration, cleaning solvents, and contaminants. The chassis is formed from plastics, particularly injection molded polycarbonates, and includes a handle, a display screen, and a molded or sculpted recessed portion to receive at least one peripheral configured to obtain and wirelessly transmit medical data. The back of the chassis could be made of magnesium.

### FIELD OF INVENTION

The embodiments of the invention relate to a mobile computing device with integrated medical devices such as stethoscopes, blood pressure monitors, vital signs monitors, and other instruments which may be used in a clinical or hospital environment for monitoring the welfare of a patient. Illustrative of computing devices according to the present invention are tablet-style personal computers designed to communicate wirelessly with various medical testing instruments while being convenient to hold and operate and resistant to penetration by fluids.

### BACKGROUND

A class of personal computers which is smaller than the typical “desktop” or “laptop” devices are known in the art as “tablet” personal computers, or tablet PCs. Such computing devices are generally known for use as personal digital assistants and as specialized, mobile computers. Tablet PC's typically offer the advantage of a small form factor that is easy for the user to carry, but at the cost of limited utility with respect to their full-sized counterparts. As well, such devices tend to be viewed either as too fragile for industrial use or as too bulky and heavy when configured for use in harsh environments.

Further, due to size, weight, and cost limitations, manufacturers often make trade-offs when designing tablet-style PC's. Such trade-offs are usually dictated by the intended use of the PC, for example whether it will be used as a personal digital assistant, a data acquisition device, etc. Devices meant to be used outdoors have tended to appear “rugged”, which many users equate with clumsy and bulky. On the other hand, devices meant for greater portability have often been perceived by consumers to be too fragile for use under industrial conditions.

Notably absent from the prior art, as well, are devices that are suitable for medical professionals to use to gather and store physical data from patients—vital signs, photographs, etc. and store the data in a convenient, tablet-style device. Such devices should offer wireless data acquisition, data storage, and the processor capacity to run a powerful, flexible operating system in a durable, ergonomic form factor capable of withstanding exposure to liquids and other environmental factors. To accommodate the needed processing power, the device should also be capable of dissipating the heat generated by modern central processing units without causing discomfort to the user or requiring a chassis having large, inconvenient heat sinks or vents that might permit the device to be contaminated or damaged by liquids or high-humidity environments.

### BRIEF DESCRIPTION OF THE DRAWINGS

FIG. 1 is a block diagram of an illustrative embodiment of the computing hardware of the present invention.

FIG. 2 is a plan view of an illustrative embodiment of the chassis of the present invention.

FIG. 3 is an exploded view of an embodiment of the chassis of the present invention.

FIG. 4 is an exploded view of an embodiment of the chassis of the present invention showing an exemplary gasket structure.

FIG. 5 is a depiction of the inside of the chassis illustrating exemplary placements for antennae according to the present invention.

FIG. 6 is a perspective view of an embodiment of a docking station according to the present invention.

FIG. 7 is another perspective view of an embodiment of a docking station according to the present invention.

FIG. 8 is a perspective view of an embodiment of a docking station according to the present invention showing the bottom of a docking station.

FIG. 9 is a block flow diagram illustrating a possible architecture for the functioning of a still camera used in accordance with the present invention.

FIG. 10 is a block flow diagram illustrating a possible architecture for the functioning of a barcode reader and RFID scanner used in accordance with the present invention.

FIG. 11 is a block flow diagram illustrating a possible architecture for the functioning of a power button used in accordance with the present invention.

FIG. 12 illustrates an embodiment of a touchpad module.

FIG. 13 illustrates a touchpad module and block flow diagram of a possible architecture for use of a touchpad in connection with the hardware of the present invention.

FIG. 14 illustrates an embodiment of an APCI-to-HID Mapper driver architecture.

FIG. 15 illustrates an exemplary embodiment of a control/indicator area and applet.

FIG. 16 illustrates an embodiment of the present invention incorporating a stylus and recessed caddy within the chassis of a tablet-style PC.

FIG. 17 is a perspective view of an embodiment of a wireless stethoscope.

### DETAILED DESCRIPTION

As used in the specification and claims, the singular forms “a”, “an” and “the” include plural references unless the context clearly dictates otherwise. For example, the term “an array” may include a plurality of arrays unless the context clearly dictates otherwise. Further, Table 1, below, lists various acronyms and terms of art used herein.

TABLE 1

| | |
|---|---|
| ACPI | Advance Configuration and Power Interface |
| AP | Access Point |
| API | Application Programming Interface |
| ARD | Architectural Requirements Document |
| BIOS | Basic Input/Output System, the PC firmware/boot ROM. |
| BLI | Back Light Inverter |
| BT | Bluetooth |
| CMT | Centrino Mobile Technology |
| CPU | Central Processing Unit |
| DDR | Double Data Rate |
| DPST | Display Power Saving Technique |
| EBL | Extended Battery Life |
| EC | Embedded Controller, e.g., Hitachi H8 |
| EEPROM | Electrically Erasable Programmable Read Only Memory |
| FW | Firmware |
| FWH | Firm Ware Hub, the flash memory chip that contains the |
| | BIOS |
| GMCH | Graphics Memory Controller Hub |
| GPIO | General Purpose Input Output |
| HDD | Hard Disk Drive |
| HIBCC | Healthcare Industry Bar Code standard (data format for |
| | assest tagging using barcode or RFID). |
| | See http://www.hibcc.org |
| HIPAA | Health Insurance Portability and Accountability Act. See |
| | http://www.hipaa.org/ |
| HW | Hardware |
| IA | Intel Architecture. IA-32 is 32-bit architecture. IA-32e is |
| | 32-bit architecture with 64-bit extensions. IA-64 is |
| | 64-bit architecture (Itanium family). |
| ICH | Input Output Controller Hub |
| IHV | Independent Hardware Vendor |
| ISV | Independent Software Vendor |
| IDE | Integrated Device Electronics |
| KSC | Keyboard/System Controller, H8 microcontroller used |
| | for keyboard scan, battery charging, and |
| | miscellaneous system GPIO's. |
| LAN | Local Area Network |
| LVDS | Low Voltage Differential Signaling, a style of LCD panel |
| | interface |
| MCH | Memory Controller Hub |
| NIC | Network Interface Controller |
| ODM | Original Design Manufacturer |
| OEM | Original Equipment Manufacturer |
| OS | Operating System |
| PATA | Parallel AT Attachment, a style of HDD or ODD interface |
| PC | Personal Computer |
| PCB | Printed Circuit Board |
| RAM | Random Access Memory |
| RFID | Radio Frequency IDentification |
| RTC | Real Time Clock |
| SMC | System Management Controller |
| SODIMM | Small Outline Dual In Line Memory Module |
| SW | Software |
| TBD | To Be Determined |
| TPM | Trusted Platform Module |
| TPV | Third Party Vendor |
| USB | Universal Serial Bus |
| VCOM | Virtual COMmunications port, a device driver that looks like |
| | a real COM port, but has no physical COM hardware |
| | associated with it. |
| VOIP | Voice Over Internet Protocol |
| VPN | Virtual Private Network |
| WLAN | Wireless Local Area Network |
| WMTS | Wireless Medical Telemetry Service |
| WZC | Windows Zero Configuration, an automatic WLAN |
| | configuration scheme. |
| WZP | Windows Zero Provisioning, a follow-on to WZC that also |
| | provides for automatic provisioning of DSL at home or |
| | WLAN at hotspots. |

An embodiment of the invention relates to a device comprising a chassis comprising a motherboard comprising a central processing unit; the motherboard further comprising at least one graphics and memory controller and at least one input/output controller, wherein the input/output controller communicates with at least one wireless device controller; and at least one wireless peripheral device configured to acquire medical data relating a vital sign of a patient and communicate the medical data via the wireless device controller.

Preferably, the wireless peripheral device is a stethoscope, a blood pressure monitor, or a thermometer. Preferably, the wireless peripheral device is configured to use the Bluetooth wireless communications protocol. The device could further comprise a docking station configured to receive and communicate with the chassis. Preferably, the docking station comprises at least one I/O port configured to connect a peripheral device. Preferably, the chassis comprises a polymeric material. Preferably, the chassis further comprises a heat conductive material configured to dissipate heat generated within the chassis. Preferably, the heat conductive material comprises magnesium. Preferably, the motherboard is in communication with at least one RFID reader. In one variation, the motherboard is in communication with at least one digital camera. Furthermore, the device could comprise a polycarbonate chassis having a front side having a display screen. Preferably, the display screen is an LCD or a TFT display. Preferably, the LCD has a resolution of at least 640 pixels by 480 pixels and the LCD is configured to display a pixel bit-depth of not less than 8 bits. Preferably, the chassis comprises multiple parts forming seams that are gasketed to inhibit substantial penetration of liquid through the seams. The device could further comprise at least one battery mounted within the chassis. In variation, the device of this invention could have a warm swappable battery (battery can be changed out for a fresh one when system is docked).

Yet other embodiments of the invention relate to a device comprising a chassis comprising a motherboard comprising a central processing unit; the motherboard further comprising at least one graphics and memory controller and at least one input/output controller, wherein the input/output controller communicates with one or more of at least one wireless device controller, at least one RIFD reader, at least one barcode reader, at least one USB port, and at least one digital camera; at least one wireless stethoscope configured to acquire and digitize medical data relating the heart or lung function of a patient and communicate the medical data to the wireless device controller and/or to a listening device; and wherein the chassis has a front side having an LCD.

Preferably, the stethoscope is configured to communicate using the Bluetooth wireless communications protocol. Preferably, the stethoscope is configured to transmit digitized sound to the wireless communications controller in communication with the motherboard. Preferably, the wireless communications controller is configured to transmit the sound data via an I/O controller hub to an audio subsystem and the audio subsystem is configured to convert the audio data to analog format which is capable of being emitted in audible form via a piezoelectric speaker. The docking station could comprise at least one external display adapter port such as a DB-15 connector, an HDMI connector, a DVI connector, an S-video connector, a VGA connector, or component video connectors. These could be more peripherals than those mentioned above. In one embodiment, the device of the invention had 4 USBs and an Ethernet wired LAN connector.

Other embodiments relate to a method for acquiring medical data, comprising a chassis comprising a motherboard, wherein the motherboard comprises a central processing unit, a graphics and memory controller hub, an I/O controller hub, a memory module, a data storage device, and at least one wireless device controller; providing a wireless medical data acquisition device; placing the medical data acquisition device in close proximity to a patient; monitoring a vital sign of the patient and acquiring vital sign data; and transmitting the vital sign data from the wireless medical data acquisition device to the wireless device controller.

In one embodiment, the invention is a portable computing platform for use by medical professionals. The platform is characterized as a tablet-style personal computer having a sealed chassis that is resistant to liquid penetration, cleaning solvents, and contaminants. The chassis is formed from plastics, particularly injection molded polycarbonates, and includes a handle, a display screen, and a molded or sculpted recessed portion to receive at least one peripheral configured to obtain and wirelessly transmit medical data. The magnesium back could also be part of the chassis.

The invention may also include a digitizer positioned behind the display screen and stylus, to permit data entry by a user. Peripheral devices and features that might be incorporated into the platform include, but are not limited to, digital cameras, RFID readers, bar code readers, Bluetooth connectivity, audio I/O, a docking station which may include a receptacle for recharging warm-swappable batteries. The device could have the swappability of battery pack feature.

The invention, in various embodiments, might also include wireless medical data acquisition devices such as stethoscopes, blood pressure monitors, thermometers, and other devices which acquire patient-data that a medical professional may wish to record directly into a computing device.

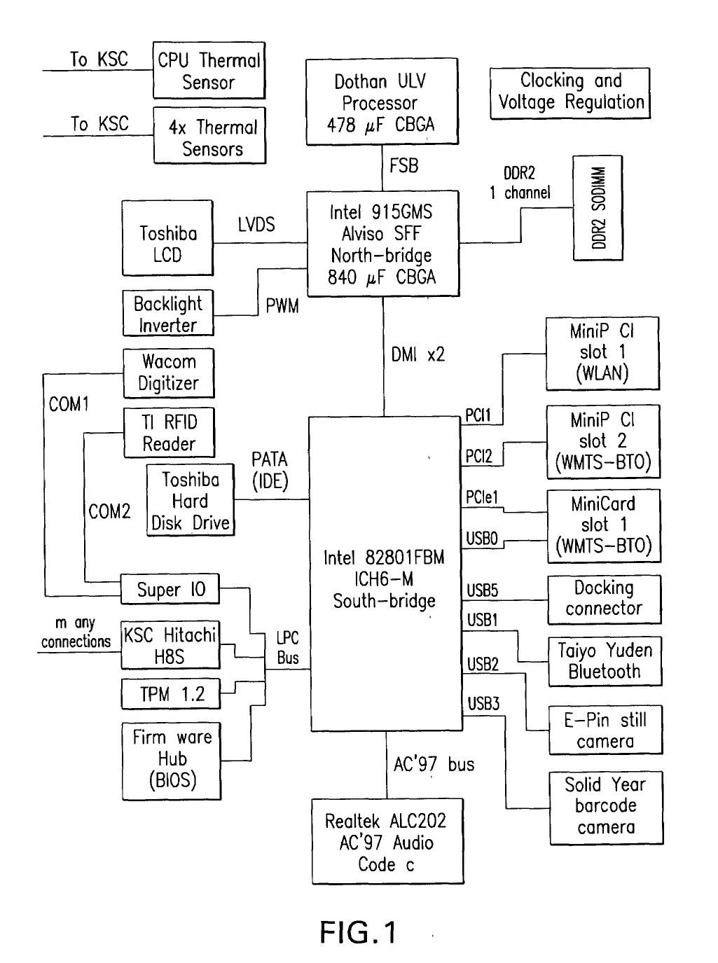

An illustrative embodiment of the computer hardware component of a tablet PC according to the present invention is shown in FIG. 1. As shown, the motherboard includes a CPU. In this embodiment, the illustrative CPU is a 478-ball Dothan ULV processor in a Micro-FCBGA package. The CPU may be soldered down to the motherboard or it may be socketed to facilitate the replacement of defective units, to permit the end-user to upgrade the processor, etc. The selected CPU runs nominally at 1.2 GHz in High Frequency mode and at 600 MHz in Low Frequency mode. Other CPUs may be used, although size, heat dissipation, and power requirements may change in other parts of the system. Those skilled in the art will recognize and be able to adapt hardware aspects that must be accommodated for other processors.

Faster processors, for example, may be larger in size and generate more heat while consuming more power; and smaller processors may require less power and generate less heat that must be removed from the system. The selected CPU can also supports enhanced technologies for voltage and frequency scaling.

The system memory may be determined based on the intended application of the tablet through the use of commercially available memory modules. The illustrative embodiment of FIG. 1 may contain a single-channel, 400 MHz DDR2 capable SODIMM socket. A default configuration employing one DRAM module of 1 GB size can accommodate a wide-variety of applications, although larger and smaller capacity DRAM modules are available and can be installed at the time of manufacturing. In applications where the memory is not hard-wired to the motherboard and the tablet chassis permits opening by the user, the user or technician may be provided with the ability to change memory modules to replace defective units or increase memory capacity.

A variety of commercially available system clocks may be employed, as well. The present exemplary embodiment employs the CK-410M Clock Synthesizer. This clock synthesizer creates system clock signals that are distributed to synchronous devices within the system. The GMCH may provide the spread spectrum clock to GMCH's LVDS interface.

In communication with the processor via the motherboard's front-side bus might be the GMCH, or “North Bridge” as it is commonly referred to in the art. The GMCH (graphics and memory control hub), functions can be provided by the Alviso SFF graphics/memory controller hub model “915GMS”. This device is packaged in an 840-ball, 27 mm×27 mm Micro-FCBGA package and is usually soldered to the motherboard. It may, in some instances, be useful to provide the GMCH in a socketed configuration, if convenient substitution of the unit is desirable.

Video capability may be provided via a TFT LCD, or other flat-panel display that can be incorporated into the chassis of the device. In the present embodiment, only a single display is required, thus the analog TV-Out and digital SDVO outputs can be disabled on the graphics controller. Only the LVDS interface to an LCD panel, or other, is necessary. Other outputs may be enabled and external ports may be provided, with adequate safeguards taken to avoid increasing the risk of liquid incursion into the chassis, in instances where external video is desirable.

The ICH, also referred to as the “South Bridge”, provides I/O capabilities. In one embodiment of the present tablet, these services are provided by the ICH6-M I/O controller hub (ICH). This ICH may provide a DMI interface to the “North Bridge”, a PCI Bus which can be routed to one or more MiniPCI card connectors, a PCI Express (PCIe) Bus which may be routed to one ore more MiniCard card connectors, a PATA interface for providing a data path to a hard disk drive, and a SATA interface. The SATA interface may or may not be used, depending on the number and types of data storage units required. Other storage device interfaces may be used, as well, if a different ICH is selected for use in the tablet.

The ICH can also provide USB ports. The ICH6-M provides Eight (8) USB ports for devices such as cameras, barcode readers, Bluetooth wireless communications controllers, docking connectors, etc. Other ICHs may provide more or less, depending on the needs of the system and the anticipated number of USB peripherals. As well, the ICH should provide an audio bus, to provide the device with sound capability. The ICH of this illustrative embodiment is configurable for AC'97 or Intel High-Definition Audio.

The ICH can also provide a LPC (Low Pin Count) Bus. The LPC bus may connect to the firmware hub, i.e., the Flash EPROM storing the BIOS code and support the use of a KSC (in this embodiment a Hitachi H-8 Keyboard/System Controller). The LPC may also provide a communications path to a Super-I/O chip with two RS-232C serial ports; and a TPM (Trusted Platform Module) chip that provides security key storage. Variations between available ICHs may permit different hardware to be connected to the system via the Southbridge to accommodate varying hardware configurations.

The system may also employ thermal sensors to permit monitoring of thermal conditions within the chassis and for various components on the motherboard. Most modern CPU's, such as the one employed in the exemplary embodiment, include an on-die thermal sensor. Further, an external thermal diode positioned very near the CPU package can be connected to a remote thermal sensor such as Analog Devices ADM 1023. The remote thermal sensor's SMBus may interface with the KSC's SMBus and also to the processor's “Critical Thermal” pin. When the “Critical Thermal” pin is driven, the processor is designed to perform an emergency shutdown. Typically, when such a shutdown occurs, the operating system state will not be saved. Prior to that event, the current temperature can be read via the KSC, and the KSC may also be programmed to provide a warning interrupt when a temperature threshold (also called a “thermal trip point”) is crossed. Additional sensors may be employed to increase the level of monitoring or for system-design debugging purposes.

System power may be provided by an internal system battery pack or by mounting in a dock which provides an integral AC/DC converter “brick”. The battery pack can be charged through an on-board charger using, for example, a MAX8724 chip (a battery charger controller) controlled by the KSC. The charger may then used to charge and control the batteries and provide system regulation of +12.6 VDC when external power is provided. When only battery power is available the voltage may typically range from a maximum of about +12.6 VDC (fully charged) down to a minimum of about +9.0 VDC (at discharge cut-off). Different power supply schemes, of course, may result in variations of the minimum and maximum voltages.

The power from the two paths described above is typically input to most of the on-board voltage regulator circuits to provide power to all system components. The input voltage may be converted by various commercially available components to provide a variety of rail voltages. In the present embodiment, a 4-in-1 controller (e.g. TPS5130) may be employed to develop the system voltage rails including, at least, +5.0V, +3.3V, +2.5V, and “+1.5V ALWAYS”. Other voltage rails may be developed and supplied to peripherals and system hardware requiring, based on need, by employing appropriate voltage controllers.

Typically, chipset and memory subsystems require separate regulation to provide +1.8V, +0.9V, and +1.05V. This power may be provided by two dual regulator circuits with one providing the memory supply and the other the VCCP (CPU Core) and GMCH core power.

Various “always” power rails may be switched using FETs (Field Effect Transistors) to provide switched rails when system S-states require power to be controlled on or off at various times.

As well, an IMVP-IV (Intel Mobile Voltage Power) solution from Analog Devices for CPU core voltage regulation may be provided. On the Dothan ULV, six VID outputs control the voltage during C0-C3 states. The C4 state (“Deeper Sleep”) is controlled by the ICH6-M's DPRSLPVR (deeper sleep voltage regulator) signal and a precision resistor.

According to the present, illustrative embodiment, six (6) Lithium prismatic cells, such as the Panasonic CGA103450A, are bundled into a single battery pack in a 3S2P geometry. A charging controller board may also be included in this package. Each cell typically provides 1950 mAh of storage nominally at 3.7 VDC, for a total pack capacity of 3900 mAh at 11.1 VDC. This works out to approximately 43 Wh.

At an average and continuous system consumption rate, for example about 12 W, a battery life of approximately 3½ hours on a single charge may be anticipated. When the system is in suspend or hibernate modes, battery life will be extended. During times of heavy use (complex computation), battery life will shorten.

The dock may also contain a charging cavity for a second battery, which can be “warm swapped” (exchanged without powering down the system) with a discharged battery while the tablet is stationed in the dock and receiving A/C power.

An A/C power “brick” is typically provided to provide electrical service to the dock. The A/C power brick may be one such as the Powertron Electronics Corporation model F10653-A. This pack is designed to connect to the wall source power on one end, and the docking cradle on the other end. Such a brick may accept input at 110 VAC to 240 VAC from 47 Hz to 63 Hz, so should be usable worldwide, assuming the correct physical adaptor plug is used. Typically, units manufactured for the North American market might be supplied with a 3-prong (grounded) plug. Other plugs, of course, can be can be used to accommodate power outlet configurations used elsewhere in the world. This brick could be placed inside of the dock, so the only thing coming out of the dock to support this brick could be a power cable.

Illustratively, the power brick may provide output at +19 VDC +/−5% at a maximum of about 3.42 A and have a barrel-type plug with positive voltage on “tip” and ground on “ring”. Other styles of power bricks to provide for other voltage requirements, lower voltage tolerances, and higher or lower anticipated current requirements are known in the art.

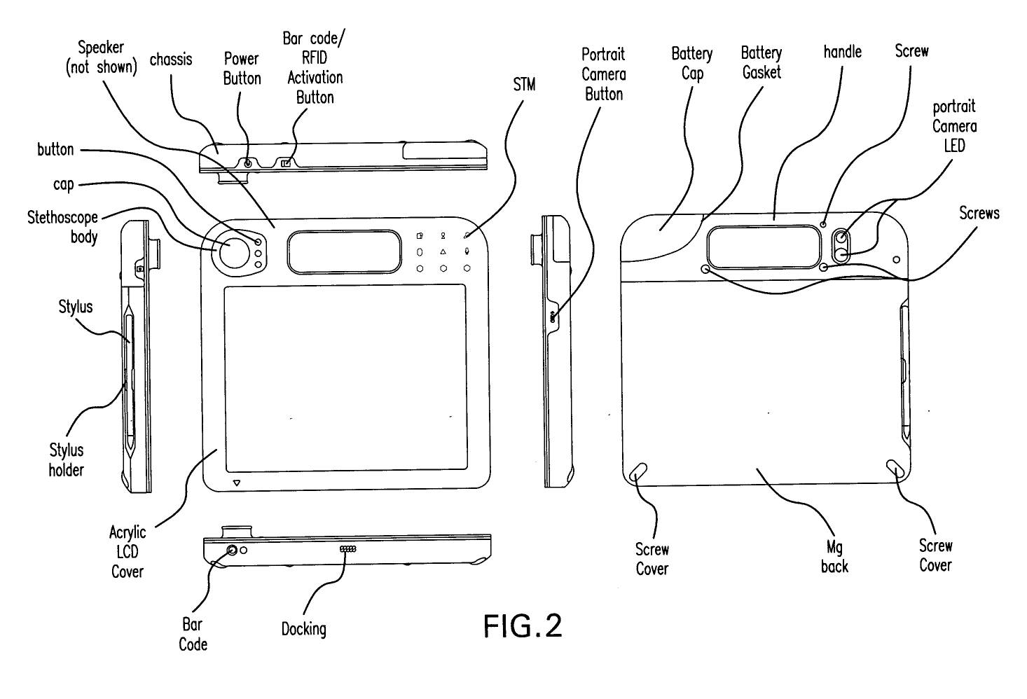

The tablet PC of the illustrative embodiment, as illustrated in FIG. 2, may be a thin and light design targeted to the healthcare vertical market segment and tailored to predicted usage models primarily by nurses and secondarily by doctors. Key system design features might include a rugged, rounded, professional appearance and a sealed chassis resistant to bio-fluids and germ growth. The chassis should be constructed in a manner permitting it to withstand cleaning using anti-bacterial reagents. It is also desirable to provide an ergonomic layout with carry handle and peripheral positioning.

When used to provide a table-style PC for use in hospitals or a clinical environment, the chassis may be designed to provide for the integration of technological features such as a stethoscope, vital signs monitoring equiment (temperature, blood pressure, etc.), or other peripherals desired for medical professionals. In one embodiment, these devices communicate wirelessly with the tablet via Bluetooth, 802.11 wireless protocol, or other wireless data transmission protocol.

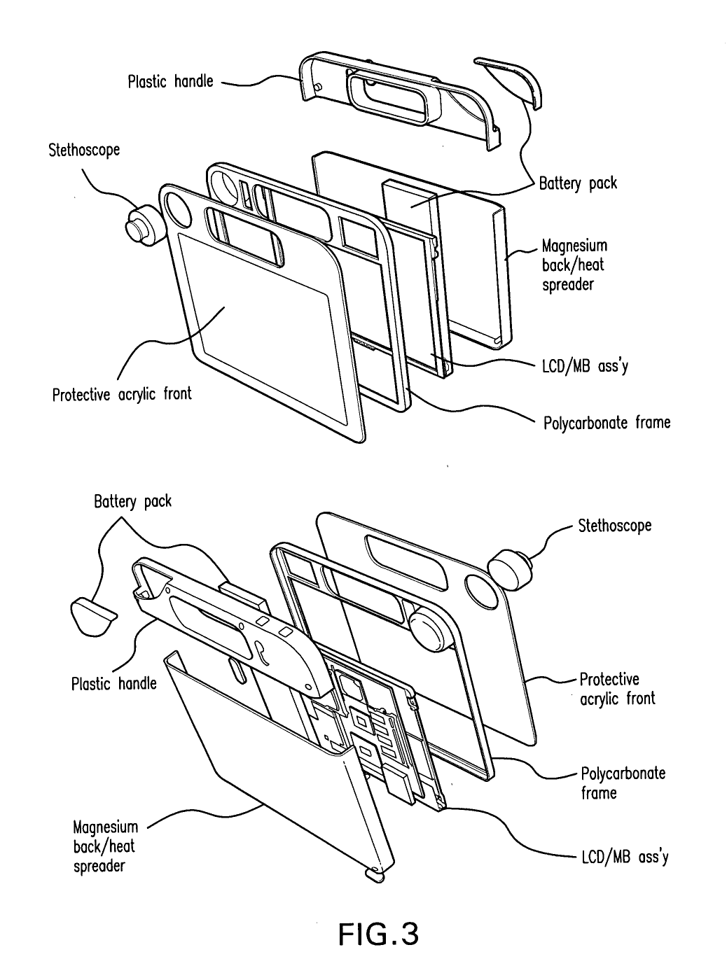

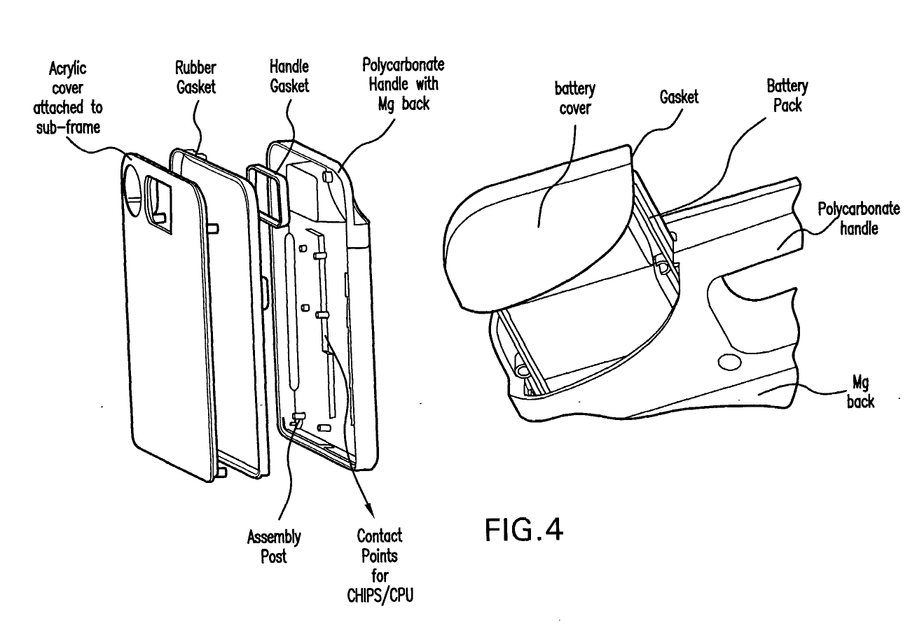

The chassis may comprise the following components, illustrated in FIGS. 3 and 4: a front acrylic protective plate; polycarbonate underlay frame, an electronic assembly for the LCD or other display screen, a digitizer, a motherboard and daughter-cards. The device may also comprise a back plate which includes a plastic carry-handle assembly and a heat spreader/sink. In order to provide adequate sealing provide a unit with resistance fluid penetration, it may be desirable to provide a back plate that includes a flat-plate comprised of a metal that dissipates heat quickly, such as magnesium, titanium, aluminum, copper, etc. The heat spreader plate may, as well, be in physical contact with motherboard components that require heat dissipation, such as the CPU, although such contact may be made via substances such as thermal grease or intermediate layers of heat-conductive metal. This permits the back-plate of the tablet to act as a heat-sink, thereby avoiding the need for internal fans or other means for heat removal that may compromise the unit's ability to resist penetration by fluids, moisture, and other contaminants.

In the illustrative embodiment, the battery pack may incorporate a plastic cap that provides a mating seal to the handle assembly. A stethoscope, or other peripheral, may be formed to insert into a void in the acrylic surface and rest in a cavity sculpted or otherwise formed in the polycarbonate frame. The construction materials, of course, are merely presented for purposes of illustration; those skilled in the art will recognize that a wide variety of metals and plastics may be substituted for any of the chassis components, provided that issues with magnetic and electrical shielding for the components and various antennae are accounted for.

To provide a chassis that is well-sealed to be water/fluid resistant and resistant to cleaning with industrial chemical solvents, or other materials, the chassis components may be assembled with interposing rubber o-ring gaskets, or similar gaskets able to provide fluid-resistance for each of the seams where chassis components meet.

All seams in a system designed to be fluid resistant should generally be gasketed to prevent liquid penetration into the system. A main gasket that seals the top and bottom subassemblies would typically be provided, therefore. This gasket also integrates the “hard” buttons (power, camera shutter, barcode/RFID scan) to provide sealed button actuation, where buttons or button pads are employed. Further, any internal gaskets that will seal the handle area may be provided in a manner that is not visible to the user.

The battery cap will contain a rubber diaphragm that will form a compression seal against the plastic handle area. All fasteners will use o-rings or silicon for sealed assembly.

In order to achieve a high degree of thermal performance the primary components may be cooled by .the integrated chassis/heat sink. For example, the chassis may be made of injection molded magnesium frame, or other suitable highly heat conductive material. The frame may then be coupled directly to the CPU, MCH, and ICH or indirectly via thermal grease or intermediate layers of heat conductive material.

A thermal shield may be implemented over the top of the chassis/heat sink to limit the heat transfer rate from the heat sink to the user. No fans or system vents are integrated to maintain sealability of the system in instances where liquid penetration is a feature of the tablet.

According to the structure describe above, the system is passively cooled. Heat is transferred out of the system via conduction, natural convection, and radiation. An insulative shield may be applied to the back of the display screen, for example an LCD, to maintain its required ambient temperature and provide a more uniform temperature profile across the surface of the display.

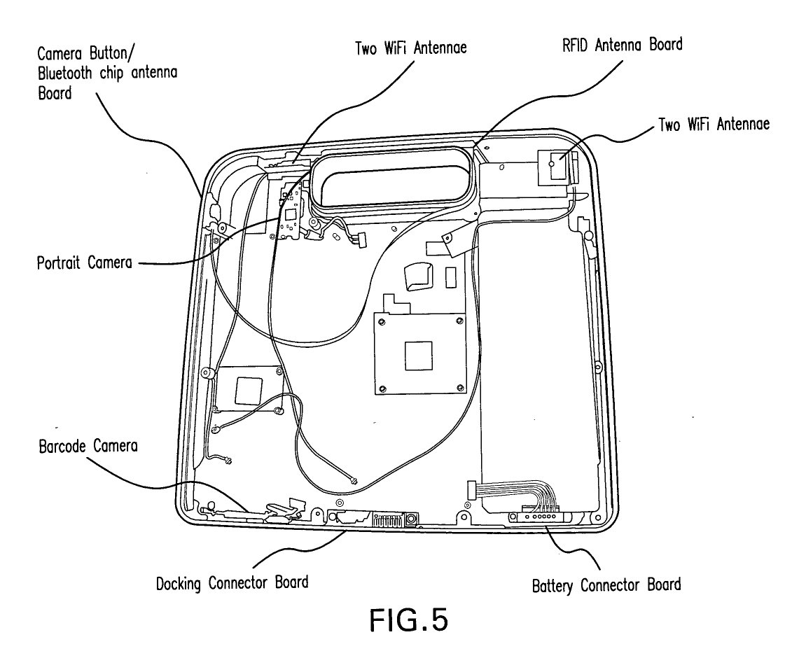

FIG. 5 illustrates an exemplary placement of the antennae, cameras, and daughterboards in a chassis. Design criteria for component placement may include factors such as magnetic and electrical shielding, thermal shielding or dissipation, RF interference, space constraints, and ergonomics. This list, however, is merely illustrative and not exhaustive of the considerations necessary for component placement; and no single solution may necessarily better than others.

As previously mentioned, the device will ordinarily include a display, such as an LCD, TFT, or other light-weight, portable display. The illustrative system uses an AND Displays 10.4″ inch color TFT/LCD Module, model ANDpSi104EA5S-HB. This display supports XGA (1024(H)×768(V)) screen resolution and 262K (RGB 6-bits data driver) or 16.7M (RGB 8-bits data driver) color depths. The input signals are LVDS interface compatible and it uses a single side-firing CCFL backlight.

Power consumption is 3.7 W typical (using standard SMPTE test pattern) when running at full intensity of 180 nits (cd/m2). Power consumption at 60 nits is 2.87 W. The LCD Display, a digitizer, and motherboard may be mated as a single assembly, and shock-mounted to the chassis. The system may also include a backlight inverter.

The system design includes a DB-15 connector for VGA external display connection, but will ordinarily be unused, as VGA connectors are not sealable. The connector is typically not stuffed on motherboards that are assembled into a chassis, but users requiring external video may desire a tablet device that offers this feature.

The system may also incorporate digitizer. In the illustrative embodiment, the digitizer is a Wacom SU-001-A 10.4″ diagonal electromagnetic (inductive) digitizer that underlaps the LCD. This digitizer has a true resolution accuracy of 0.001 mm (2540 dots/inch) and may report up to 133 points/second during stylus motion. This digitizer meets Windows XP Tablet Edition requirements, thereby making it usable in instances where said operating system is employed.

The system may also be equipped with a stylus, to permit data entry directly into the device via the digitizer. In the illustrative embodiment, the stylus is passive. A suitable stylus device includes the Wacom “Penabled Tablet PC Slim Pen”, model MP200-00 that is 5.5 mm in diameter. The pen can report 256 different levels of pressure when the stylus is pressed against the acrylic LCD protector. The stylus can be sensed at distances between 5 mm and 14 mm away from the digitizer board (this includes the thickness of the LCD panel, air gap, and a protective acrylic cover).

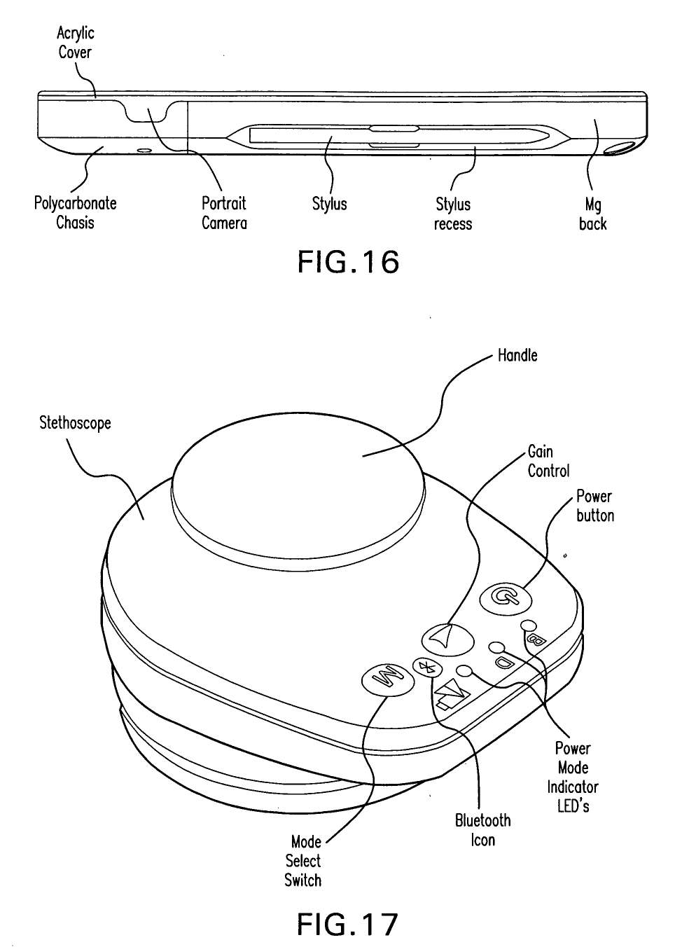

The system may accommodate the stylus in a recessed caddy area, as shown in FIG. 16. When using a passive device, there are no electrical connections related to the stylus (e.g., no “removal” indication to software).

To facilitate data and software storage, the system may contain at least one mass storage device, such as an integrated hard disk drive (HDD). Illustratively, the HDD may be a Toshiba 20 Gigabyte 1.8-inch diameter drive, model MK2008GAL. This HDD uses PATA as the interface to the baseboard. There is typically provided a PATA connector directly on the on the baseboard that may be used for a ribbon-cable connection to the “CE” style connector on the HDD. This drive is 5.0 mm thick, making it suitable for use in a portable device such as a tablet PC.

The illustrative embodiment of the tablet may include a wireless LAN subsystem. This may consist of a MiniPCI connector on the motherboard, with a WiFi card installed. Suitable for use is the commercially available Intel PRO/Wireless 2915 ABG. It supports the IEEE industry standards 802.11 a, b and g.

Certain peripheral devices may be connected to the tablet via wireless LAN or Bluetooth technology. The present device may, therefore, also incorporate a Bluetooth controller such as the Taiyo Yuden EYSFCCSXX module, to provide Bluetooth capability for the system. This device incorporates the CSR (Cambridge Silicon Radio) “Bluecore 4” radio chip, operating in the 2.4 GHz band. The module implements Bluetooth 2.0 specifications, and includes AFH (advanced frequency hopping) and EDR (enhanced data rate) functions. The module interfaces to the system using one of the USB ports available via the ICH.

The Taiyo Yuden EYSFCCSXX module also supports WiFi coexistence “Phase 2” capability. This capability embodies arbitration for the 2.4 GHz spectrum between WiFi and Bluetooth radios. This reduces the interference between the Bluetooth and the WiFi radios when they are operating simultaneously. The two modules have a communication channel that they use to inform one another about when they are transmitting, and what WiFi channel is being used. The Bluetooth module attempts to choose a different channel in the 2.4 GHz band which does not conflict with the WiFi channel in use (determined by access point association).

The WMTS subsystem may also include a “dual stuffing option” connector layout on the motherboard. The motherboard, therefore, may contain contacts (“pads”) for both MiniPCI and MiniCard (aka Mini-PCI Express) socket connectors. These pads are designed to use substantially the same physical volume inside the system.

An OEM employing this feature would determine, at manufacturing time, which connector to solder to the motherboard, since most compact chassis layouts will permit only one can be used at a time. Then the OEM may insert the appropriate form-factor WMTS card into the system before sealing the chassis.

In instances where the tablet user will benefit from having an RIFD reader incorporated into the device, a suitable hardware solution may include the Texas Instruments (T.I.) 7961 RFID reader chip and companion MSP430 microcontroller. This device may be connected via an RS-232 interface at TTL levels (i.e., +5 VDC and Ground, vs. the more conventional +12 VDC and −12 VDC) to the COM2 port of the Super-IO. This T.I. chip supports RFID protocols ISO 15693, ISO 14443, and T.I.'s “Tag-It”.

The RFID reader is a relatively low power device and has a short reading range on the order of 4 to 5 centimeters. The antenna may be positioned in the plastic handle area (it should be as far away from any metal as reasonably possible, to read effectively). As a result, the user should position the RFID-tagged object near to the antenna location for scanning.

An audio subsystem may be incorporated into the device, to provide sound output. One suitable device is based on a Realtek ALC202 codec, which is compliant with the AC'97 specifications. The system may also contain an internal power amplifier to more effectively drive the internal speaker. Exemplary of such amplifiers is the LM4960SQ. Another exemplary amplifier is Maxim MAX9713. One of the features of MAX9713 is driving the voltage needed by a piezo, and supported bridge mode output. A single mono speaker, a custom-designed piezo-electric transducer, can provide audio output. The transducer may be mounted to the back of the display screen protector in the area between the medical peripheral slot (stethoscope) and the handle cutout. If higher-quality audio output is desired, a Bluetooth headset may be used, to avoid the need to add I/O ports to the chassis. For that same reason, the illustrative embodiment employs a microphone input via a Bluetooth headset, or instances where live sound input is desired.

In order to avoid increased risk of penetration by liquids and contaminants, the table will generally not include externally accessible audio I/O jacks; as such jacks would create difficulties in maintaining the sealed nature of the system. If for any reason the end user requires external audio jacking, a USB audio device (e.g., Creative SoundBlaster Audigy 2 NX) may be installed into one of the free USB ports in the dock.

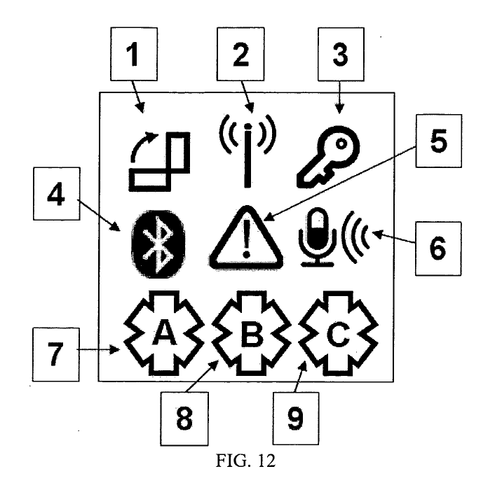

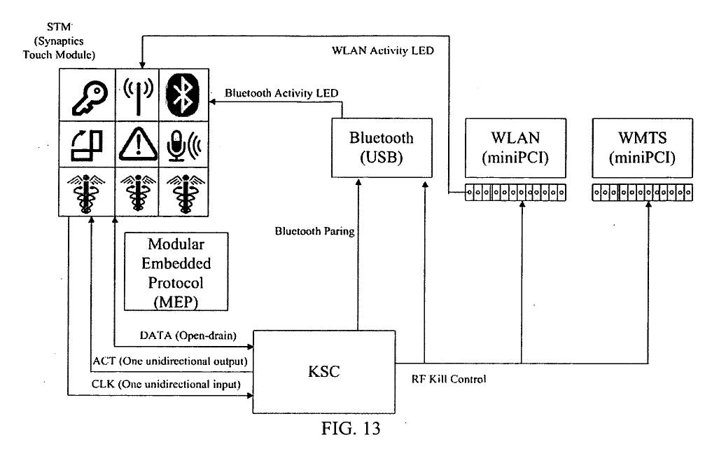

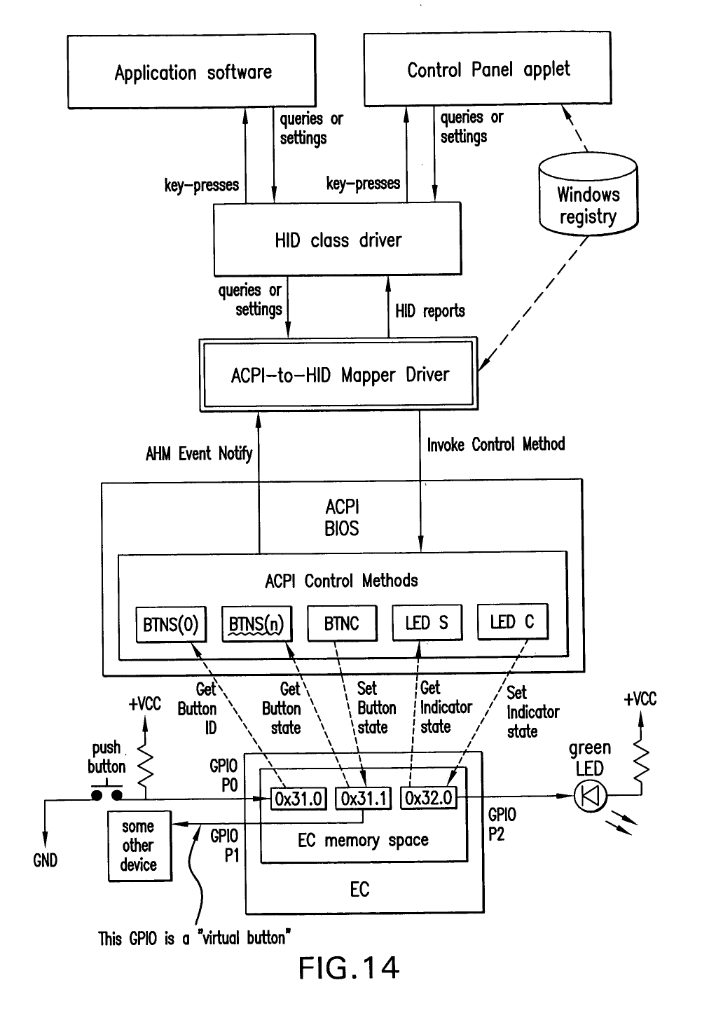

The system integrates a number of buttons and indicators, the functions and features of which are illustrated in FIGS. 12-14. Each button is assigned a “button number” which refers to the button ID assigned by the KSC. This number may be used by the KSC to report button presses to an ACPI-to-HID mapper driver. This driver may then translates the button press into an HID code for further processing. The system may include “soft” buttons. A “soft” button is one managed by the Synaptics Touch Module (STM); there is no tactile feedback from these capacitive buttons. A “hard” button is a physical momentary switch that includes tactile feedback. A “virtual” button does not have a user-accessible physical existence; it is only a software-controllable abstraction of a GPIO signal that can be driven by the KSC.

The “STM” refers to the Synaptics Mobile Touch Module, which was custom designed having a form factor specific and may be used in accordance with the illustrative embodiment of the invention. The STM enables button use without actual physical contact with the buttons; this supports the sealed system design. Other button-handling solutions will be recognized by those skilled in the art. The STM may contains both capacitive buttons and LEDs integrated into a single package. The STM interfaces to the KSC using a “MEP” protocol defined by the manufacturer.

The illustrative device will generally include a power button that is used to turn the system on and off, and also to put a running system into sleep or hibernate modes (per Windows Control Panel configuration settings, when a Windows O/S is used). The KSC monitors the user press of the physical button and sends onward the appropriate signal to the power and voltage regulation circuitry. In addition, the KSC monitors the CPU state as represented by status pins on the ICH, and may reflect the appropriate status condition on a power LED.

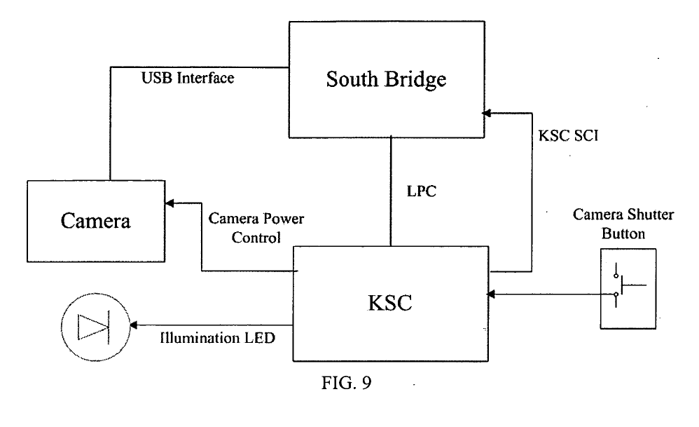

A shutter button may be used to activate the still photo applet, as illustrated in FIG. 9, and to take photo snapshots, when a camera-type peripheral is incorporated into the tablet. Typically, KSC will monitor the user press of the physical button and sends an SCI interrupt signal to the ACPI framework. This may be delivered to the ACPI-to-HID Mapper driver which translates it into the appropriate HID button code. The camera applet can respond to the HID code by instructing the KSC to power-up the camera, turn on the white illumination LED, and then over USB instructs the camera to take the snapshot. Afterwards, the applet instructs the KSC to turn off the illumination LED and power-down the camera.

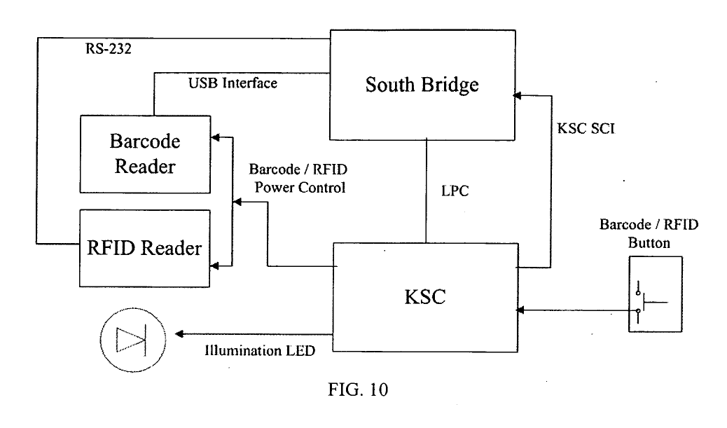

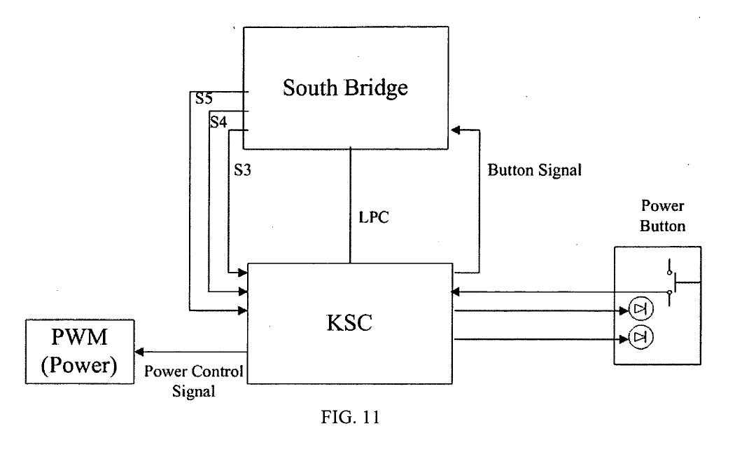

A scan button may be used to activate the applet to perform barcode and RFID scanning, shown in FIG. 10. For example, the KSC might monitor the user press of the physical button and send an SCI interrupt signal to the ACPI framework. This is delivered to the ACPI-to-HID Mapper driver which translates it into the appropriate HID button code.

The applet may responds to the HID code by instructing the KSC to power-up the camera and RFID readers, turn on the white illumination LED, and then via USB instruct the camera to grab image frames for barcode analysis and decoding. Simultaneously, via an RS-232 interface, the RFID reader may be instructed to begin searching for nearby RFID tags. When either one of the barcode decodes or RFID scanning functions returns a successful result, the applet may instruct the KSC to turn off the illumination LED, power-down the camera, and the RFID reader.

Synaptics Touch Module (STM) soft buttons may be used for various system management functions. The STM can contain an embedded microcontroller that interfaces to the KSC using a “MEP” (Modular Embedded Protocol) interface. When the STM reports a button press to the KSC, the KSC sends an SCI interrupt signal to the ACPI framework. This is delivered to the ACPI-to-HID Mapper driver which translates it into the appropriate HID button code. An applet responds to the HID code by performing the appropriate function, and in some cases, instructing the KSC to turn on or off specific LEDs by forwarding the command to the STM. In one embodiment of the invention, wireless activity status LEDs (for either or both WiFi and Bluetooth) are driven to the STM directly by the wireless card(s) rather than via the KSC. A performance advantage may be gained in this manner.

For security, a device such as the Infineon SLB9635TT TPM may be used to store credentials securely on the notebook. This device is packaged in a 28-pin TSSOP package, and connects to the ICH via the Low Pin Count (LPC) bus. It is compliant with TPM 1.2 specifications.

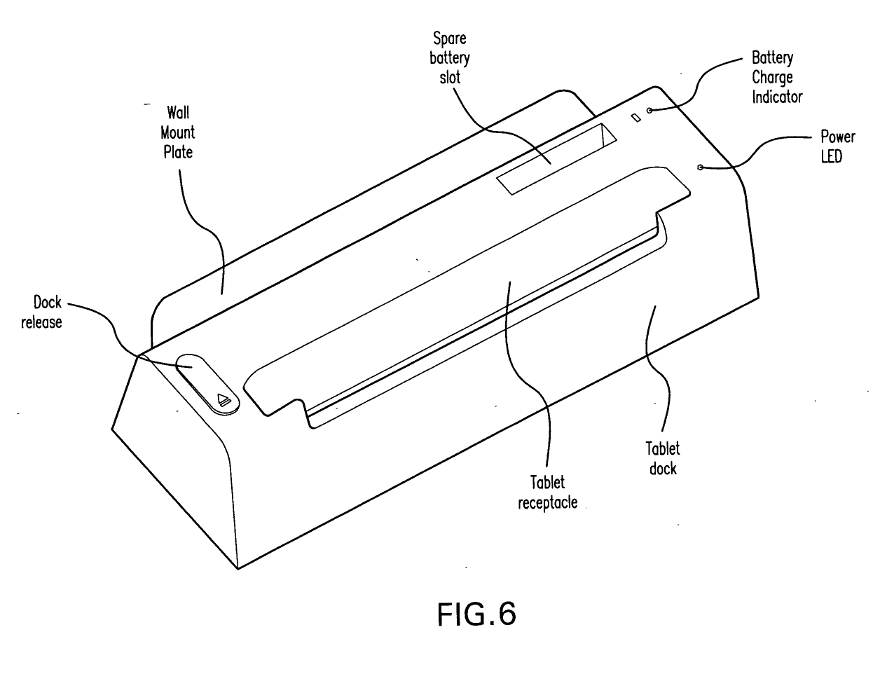

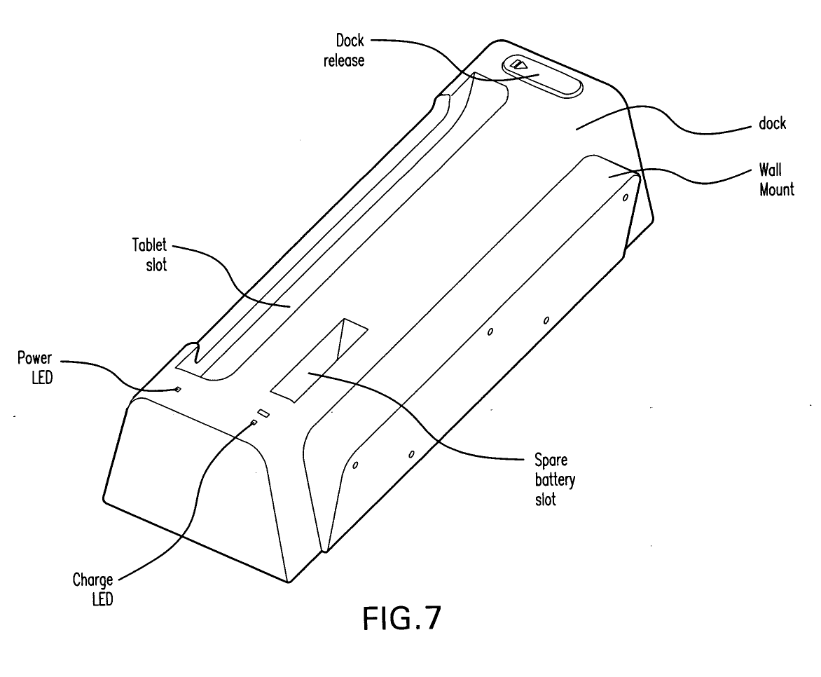

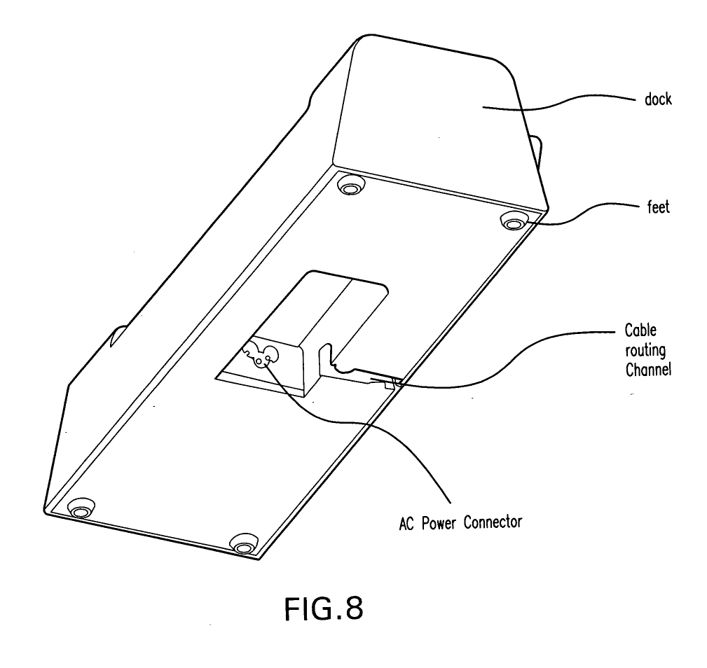

The tablet device user will typically desire the user of “dock” for the tablet device. The device may be inserted into the dock to recharge the batteries and to add additional functionality to the device via additional I/O ports, external graphics ports, etc. In one embodiment, illustrated in FIGS. 6-8, the dock houses the tablet in a manner that allows it to stand upright and still have the screen be completely viewable. It might include battery charging contacts for the tablet, as well as a charging cavity for a spare battery. LED indicators can be provided to communicate charging status.

The dock may contain a USB hub (presently, USB 2.0 is the most common solution). USB functionality may be implemented with a device such as the Philips ISP1520 USB controller chip in an LQFP64 package, which is a commercially available package. The hub chip has 1 upstream port (goes to the docking connector) and 4 downstream ports. Of the 4 downstream ports, 3 of them are exposed as external USB “type B” sockets. The final downstream port connects to the Ethernet chip. The hub supports USB2.0 data transfer at high-speed (480 Mb/s) and at legacy (USB 1.1) full-speed (12 Mb/s) and low-speed (1.5 Mb/s) rates.

The dock may also contain an Ethernet (IEEE 802.3) interface. The Ethernet functionality might be implemented with the Asix AX88772LF chip in a LQFP128 package, which is a commercially available package. The Ethernet chip, desirably, contains both MAC and PHY in a single package, and supports USB2.0 and 802.3 operations at 100 Mb/s and 10 Mb/s. The Ethernet interface may be available on the dock via an external RJ45 socket. A docking connector, in the figures shown as a flush-mounted, injection molded port, provides power and USB connectivity between the tablet and the dock. The dock will, generally, also include necessary A/C power components and cabling.

In the illustrative embodiment, the device includes at least one peripheral data-acquisition device for use by healthcare professionals. Such a device might include a Bluetooth-enabled stethoscope, as shown in FIG. 17, for use by a clinician to examine heart and lung sounds of patients. The stethoscope may include a rechargeable battery (or non-rechargeable battery) and be capable of transmitting audio-output directly to a headset worn by the user (typically the headset will also be Bluetooth-enabled). Alternatively, the stethoscope may transmit to the tablet and the tablet could retransmit the audio output to a headset worn by the user or play the sound via the tablet's internal audio system and speaker.

The stethoscope may include numerous buttons and indicators to permit the user to change the audio output device, set the type of measurement being taken, and perform basic functions (such as turn the device on and off). The stethoscope may also transmit information to the tablet relating to battery level, include audio filters to permit more accurate audio representation of a patient's heartbeat, lung function, etc., or include other functions desired by the healthcare professional. An illustrative stethoscope for use with the present tablet is shown in FIG. 17.

Those skilled in the art will readily recognize how to implement low-level software features such as the system BIOS. In the illustrative tablet, however, the BIOS may be configured to implement ACPI control methods for abstracted application control of buttons and LEDs that are managed by the KSC. Applications generally send HID messages, which are processed by the ACPI-to-HID Mapper Driver, which in turn invokes the ACPI methods in the BIOS. When a button is pressed, the BIOS generates a “notify event” to the operating system. The driver will capture this event, and call the BTNS method with an Arg0 equal to 0 to obtain the details of the button press event.

Further, the ACPI-to-HID Mapper driver may provide a system-independent way for application software to control and communicate with buttons and LEDs integrated into a notebook system that are controlled by the Keyboard/System Controller (KSC). This driver may simultaneously exist as a HID class mini-driver (for interfacing to the Windows operating system and applications) and an ACPI Driver (for interfacing to the BIOS and the KSC).

On its top interface, the Mapper driver communicates with the HID class driver to obtain HID LED setting messages (“output reports”), and to deliver HID button input messages (“input reports”). The driver reads configuration information from the Windows registry (in instances where a Windows operating system is used, as in the illustrative embodiment which might use the Windows XP Tablet Edition 2005 operating system) in order to know the HID codes that it should pay attention to (for LED settings) and the HID codes that it should generate (for button presses).

On its bottom interface, the driver registers for ACPI Events generated from the BIOS plus KSC, and invokes ACPI Control Methods to send commands to the BIOS plus KSC. The KSC (also known as the EC) actually connects to buttons and LEDs integrated into the system.

The BIOS may also implement ACPI control methods for reporting the temperature values of thermal sensors that are managed by the KSC or for reporting the power usage values of wattage sensors.

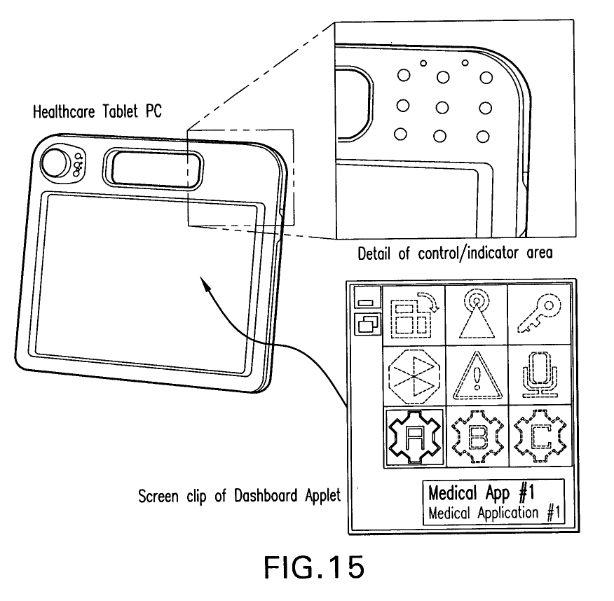

The illustrative tablet-style PC may also equipped with a touch-sensitive button and LED array that provides various system functions while still maintaining its fluid-resistant sealed design. A “Dashboard applet” may be included, as illustrated in FIG. 15, which is a Systray applet that controls the operation of this hardware by detecting button presses, performing the associated actions, and changing the state of the LEDs as appropriate. In addition, the applet provides end user mnemonic assistance in the form of on-screen “tool tips” whose visibility can be exposed or hidden.

The disclosed invention has numerous, practical embodiments. The various embodiments are to inventions useful for those requiring a portable computing platform that is durable and resistant to penetration by liquids and moisture. The device is also resistant to chemical and other cleaning solvents used to minimize the spread of germs and bacterial through contact with portable objects in a hospital, clinical, and/or other environment. While the disclosed embodiments relate generally to a portable computing platform for medical professionals, those skilled in the art will readily recognize the need for a computing platform in accordance with the present invention in a wide variety of fields.

This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A device comprising:
a chassis comprising a motherboard comprising a central processing unit; the motherboard further comprising at least one graphics and memory controller and at least one input/output controller, wherein the input/output controller communicates with at least one wireless device controller; and
at least one wireless peripheral device configured to acquire medical data relating a vital sign of a patient and communicate the medical data via the wireless device controller.

2. The device of claim 1 wherein the wireless peripheral device is a stethoscope, a blood pressure monitor, or a thermometer.

3. The device of claim 2 wherein the wireless peripheral device is configured to use the Bluetooth wireless communications protocol.

4. The device of claim 1 further comprising a docking station configured to receive and communicate with the chassis.

5. The device of claim 4 wherein the docking station comprises at least one I/O port configured to connect a peripheral device.

6. The device of claim 1 wherein the chassis comprises a polymeric material.

7. The device of claim 1 wherein the chassis comprises a heat conductive material configured to dissipate heat generated within the chassis.

8. The device of claim 7 wherein the heat conductive material comprises magnesium.

9. The device of claim 1 wherein the motherboard is in communication with at least one RFID reader.

10. The device of claim 1 wherein the motherboard is in communication with at least one digital camera.

11. The device of claim 1 comprising a polycarbonate chassis having a front side having a display screen.

12. The device of claim 11 wherein the display screen is an LCD or a TFT display.

13. A device comprising:
a chassis comprising a motherboard comprising a central processing unit; the motherboard further comprising at least one graphics and memory controller and at least one input/output controller, wherein the input/output controller communicates with one or more of at least one wireless device controller, at least one RIFD reader, at least one barcode reader, at least one USB port, and at least one digital camera;
at least one wireless stethoscope configured to acquire and digitize medical data relating the heart or lung function of a patient and communicate the medical data to the wireless device controller and/or to a listening device; and
wherein the chassis has a front side having an LCD.

14. The device of claim 13 wherein the stethoscope is configured to communicate using the Bluetooth wireless communications protocol.

15. The device of claim 14 wherein the stethoscope is configured to transmit digitized sound to the wireless communications controller in communication with the motherboard.

16. The device of claim 15 wherein the wireless communications controller is configured to transmit the sound data via an I/O controller hub to an audio subsystem and the audio subsystem is configured to convert the audio data to analog format which is capable of being emitted in audible form via a piezoelectric speaker.

17. The device of claim 13 further comprising a docking station.

18. The device of claim 17 wherein the docking station comprises at least one external display adapter port.

19. The device of claim 18 wherein the external display adapter port is a DB-15 connector, an HDMI connector, a DVI connector, an S-video connector, a VGA connector, or a component video connector.

20. The device of claim 13 wherein the LCD has a resolution of at least 640 pixels by 480 pixels and the LCD is configured to display a pixel bit-depth of not less than 8 bits.

21. A method for acquiring medical data, comprising:
providing a chassis comprising a motherboard, wherein the motherboard comprises a central processing unit, a graphics and memory controller hub, an I/O controller hub, a memory module, a data storage device, and at least one wireless device controller;
providing a wireless medical data acquisition device;
placing the medical data acquisition device in close proximity to a patient;
monitoring a vital sign of the patient and acquiring vital sign data; and
transmitting the vital sign data from the wireless medical data acquisition device to the wireless device controller.

22. The method of claim 21, further providing a portable computer that is connected to the chassis.

23. The device of claim 1, wherein the chassis comprises multiple parts forming seams that are gasketed to inhibit substantial penetration of liquid through the seams.

24. The device of claim 1, further comprising at least one battery mounted within the chassis.

25. The device of claim 13, further comprising a heat dissipation plate, and wherein the chassis is comprises multiple parts, at least one of which is fabricated from a polymeric material, forming seams that are gasketed to inhibit substantial penetration of liquid through the seams.

26. The device of claim 1, wherein the at least one wireless peripheral device is attached to the chassis.

27. The device of claim 1, wherein the at least one wireless peripheral device is separate and removable from the chassis.

28. The device of claim 1, wherein the at least one wireless peripheral device is a bar code reader or a RFID reader.
